(19) United States
(12) Patent Application Publication
(10) Pub. No.: US 2018/0284250 A1
(43) Pub. Date: Oct. 4, 2018

(54) METHOD AND SYSTEM FOR ADJUSTING AN ACQUISITION FRAME RATE FOR MOBILE MEDICAL IMAGING

(71) Applicant: General Electric Company, Schenectady, NY (US)

(72) Inventor: Steinar Bjaerum, Horten (NO)

(21) Appl. No.: 15/471,515

(22) Filed: Mar. 28, 2017

(51) Int. Cl. G01S 7/52; G01S 15/52; A61B 8/14; A61B 8/00; A61B 8/08

(52) U.S. Cl. CPC G01S 7/52034; G01S 7/52096; G01S 15/523; G01S 7/52053; A61B 8/4427; A61B 8/5223; A61B 8/54; A61B 8/14

(57) ABSTRACT

Systems and methods are provided to adjust an acquisition frame rate of a mobile ultrasound imaging system. The systems and methods receive ultrasound data representing image data sets of an anatomical structure of interest. The anatomical structure of interest includes at least one anatomical marker. The systems and methods further determine a rate of change in position of the at least one anatomical marker between adjacent image data sets, calculate an acquisition frame rate based on the rate of change in position of the at least one anatomical marker, and acquire first and second image data sets at the acquisition frame rate.

### BACKGROUND OF THE INVENTION

The subject matter disclosed herein relates generally to ultrasound imaging systems, and more particularly, to a method and apparatus for adjusting an acquisition frame rate of a mobile ultrasound imaging system.

Ultrasound imaging systems typically include ultrasound scanning devices, such as, ultrasound probes having different transducers that allow for performing various different ultrasound scans (e.g., different imaging of a volume or body). Mobile or pocket sized ultrasound imaging systems are gaining significance due to their portability, low costs, and image quality. Mobile ultrasound imaging systems may be utilized to perform various procedures that were once only accomplished in a dedicated medical facility, for example, a hospital. Mobile ultrasound imaging systems can include diagnostic tools based on acquired ultrasound images of the ultrasound imaging system. However, mobile ultrasound imaging systems have a limited power source, such as a battery. Conventional mobile ultrasound imaging systems use a static acquisition frame rate regardless of the anatomical structure being scanned. The acquisition frame rate corresponds to an amount of time for the conventional mobile ultrasound imaging system to acquire successive ultrasound images of a region of interest. Thus, the power consumption is the same regardless of any movement of the anatomical structure. For example the acquisition frame rate is the same for an ultrasound scan of a moving anatomical structure, such as a heart, with respect to a static anatomical structure, such as a kidney.

### BRIEF DESCRIPTION OF THE INVENTION

In an embodiment, a method is provided. The method includes receiving ultrasound data representing image data sets of an anatomical structure of interest. The anatomical structure of interest includes at least one anatomical marker. The method includes determining a rate of change in position of the at least one anatomical marker between adjacent image data sets, calculating an acquisition frame rate based on the rate of change in position of the at least one anatomical marker, and acquiring first and second image data sets at the acquisition frame rate.

In an embodiment a system (e.g., a mobile ultrasound imaging system) is provided. The system includes a portable host system having a controller circuit, a power supply, and a display. The controller circuit is configured to execute programmed instructions stored in a memory. The controller circuit when executing the programmed instructions perform one or more operations. The one or more operations include receive ultrasound data representing image data sets of an anatomical structure of interest. The anatomical structure of interest includes at least one anatomical marker. The one or more operations include determine a rate of change in position of the at least one anatomical marker between adjacent image data sets, calculate an acquisition frame rate based on the rate of change in position of the at least one anatomical marker, and acquire first and second image data sets at the acquisition frame rate.

In an embodiment a tangible and non-transitory computer readable medium is provided. The tangible and non-transitory computer readable medium includes one or more programmed instructions configured to direct one or more processors. The one or more processors are directed to receive ultrasound data representing image data sets of an anatomical structure of interest. The anatomical structure of interest includes at least one anatomical marker. The one or more processors are further directed to determine a rate of change in position of the at least one anatomical marker between adjacent image data sets, calculate an acquisition frame rate based on the rate of change in position of the at least one anatomical marker, and acquire first and second image data sets at the acquisition frame rate.

### BRIEF DESCRIPTION OF THE DRAWINGS

FIG. 1 illustrates an exemplary mobile ultrasound imaging system formed, in accordance with various embodiments described herein.

FIG. 2 is a system block diagram of the mobile ultrasound imaging system shown in FIG. 1.

FIG. 3 illustrates an exemplary block diagram of an embodiment of a controller circuit of a portable host system shown in FIG. 2.

FIG. 4 illustrates a swim lane diagram illustrating a method of using a mobile ultrasound imaging system to automatically adjust a frame rate, in accordance with various embodiments described herein.

FIG. 5 illustrates an embodiment of a rate of change in position of at least one anatomical marker and acquisition frame rates.

FIGS. 6A-B illustrate image data sets of an embodiment of an anatomical structure of interest at different points in time.

FIG. 7 illustrates an embodiment of a rate of change in position of at least one anatomical marker and acquisition frame rates.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Described herein are various embodiments for a mobile ultrasound imaging system for adjusting an acquisition frame rate of a mobile ultrasound imaging system. Mobile ultrasound imaging systems are portable devices that enable a clinician to provide diagnostic medical imaging outside of clinics. Mobile ultrasound imaging systems have a limited power source, such as a battery. The power consumption of the mobile ultrasound imaging system is dependent on the acquisition frame rate of the mobile ultrasound imaging system. The acquisition frame rate represents an amount of time for the mobile ultrasound imaging system to acquire two successive ultrasound images of an anatomical structure of interest. Unlike conventional mobile ultrasound imaging systems, the mobile ultrasound imaging system described herein is configured to have a dynamic acquisition frame rate based on the anatomical structure of interest. For anatomical structures of interest that are animated (e.g., move) a higher acquisition frame rate is desired such that clinical information is not lost. However, for anatomical structures that are static (e.g., not animated) a lower acquisition frame rate may be used, which may decrease power consumption. The mobile ultrasound imaging system is configured to adjust the acquisition frame rate based on movement of the anatomical structure being imaged. The movement may be determined by the mobile ultrasound imaging system by executing an image analysis algorithm to continuously calculate and/or measure changes in position (e.g., velocity) of the anatomical structure. It may be noted that the movement may be due to repositioning of the ultrasound probe by the clinician, movement of the anatomical structure of interest, movement of the patient, and/or the like. Based on this measured change in position, the mobile ultrasound imaging system is configured to determine the acquisition frame rate.

Adjustments to the acquisition frame rate adjusts the power consumption of the mobile ultrasound imaging system. For example, reduction in the acquisition frame rate reduces an amount of electrical power demanded by the components of the mobile ultrasound imaging system. Based on the dynamic acquisition frame rate, the mobile ultrasound imaging system may be configured to optimize the power consumption. Additionally, the dynamic acquisition frame rate may adjust a temperature of the mobile ultrasound imaging system. For example, during high acquisition frame rates heat is generated by the components of the mobile ultrasound imaging system processing the ultrasound data. By optimizing the power consumption by dynamically adjusting the acquisition frame rates, the temperature of the device may be reduced even after continued use. Alternatively, such as for conventional mobile ultrasound imaging systems having a static acquisition frame rate the regulatory temperature limit is reached and the conventional mobile ultrasound imaging system becomes unusable until the temperature is reduced.

The image analysis algorithm utilized by the mobile ultrasound imaging system may be defined using a machine learning algorithm. For example, the image analysis algorithm may be configured to identify a position of anatomical markers within an image data set of the anatomical structure of interest. The image analysis algorithm may be configured to continuously and/or intermittently determine a position of the anatomical markers based on when image data sets are received. Based on changes in position of the anatomical marker over time, the mobile ultrasound system is configured to determine a rate of change of the anatomical marker between image data sets. The rate of change represents the current temporal dynamics of the anatomical structure of interest. The mobile ultrasound system is configured to determine a frame rate based on the rate of change.

A technical effect of at least one embodiment includes an increased battery life and reduced operating temperature relative to conventional mobile ultrasound imaging systems. A technical effect of at least one embodiment includes optimized performance of the mobile ultrasound imaging system with minimized power consumption without user interaction.

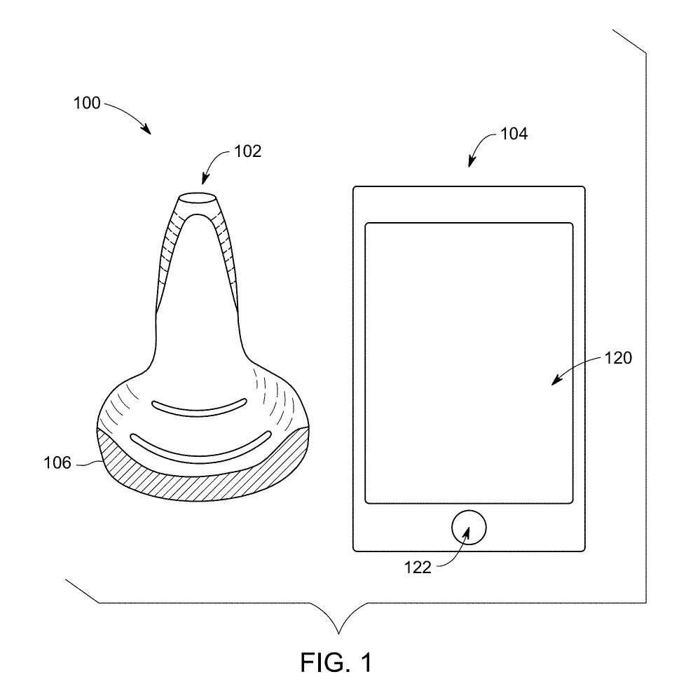

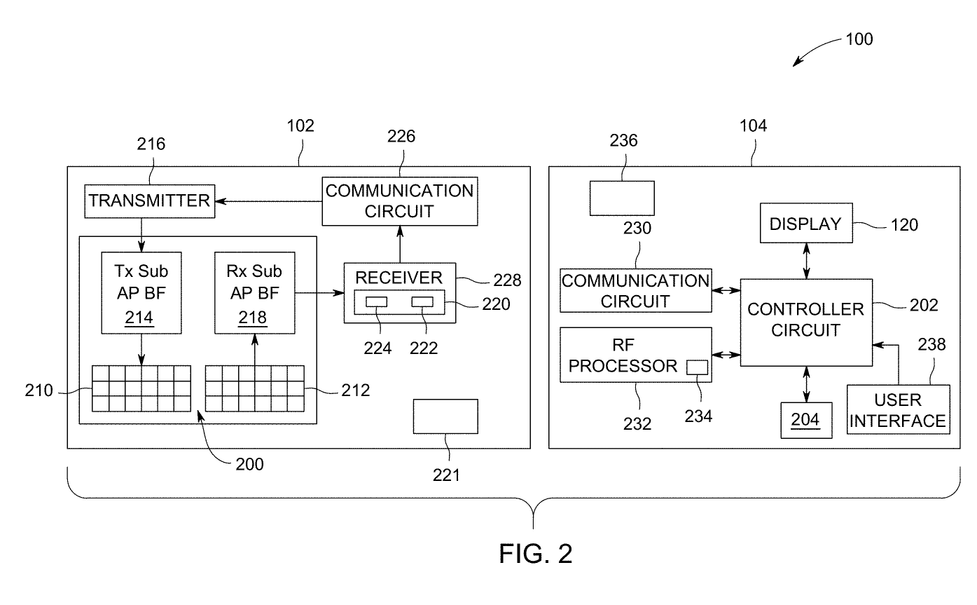

Various embodiments described herein may be implemented as a mobile ultrasound imaging system 100 as shown in FIG. 1. More specifically, FIG. 1 illustrates an exemplary mobile ultrasound imaging system 100 that is constructed in accordance with various embodiments. The ultrasound imaging system 100 includes a portable host system 104 and an ultrasound probe 102. The portable host system 104 may be a portable hand-held device, for example, a mobile phone (e.g., a smart phone), a tablet computer, a handheld diagnostic imaging system, a laptop, and/or the like. The portable host system 104 may support one or more software applications that are executed by a controller circuit 202, shown in FIG. 2, of the portable host system 104.

The portable host system 104 may include a display 120. The display 120 may include one or more liquid crystal displays (e.g., light emitting diode (LED) backlight), organic light emitting diode (OLED) displays, plasma displays, CRT displays, and/or the like. The display 120 may display patient information, one or more image data sets stored in a memory 204 and/or being acquired (e.g., in real time) and/or videos, components of a graphical user interface, measurements, diagnosis, treatment information, and/or the like received by the display 120 from the controller circuit 202.

The ultrasound probe 102 includes a transducer array 106, such as a phased array having electronics to perform sub-aperture (SAP) beamforming. For example, transducer array 106 may include piezoelectric crystals that emit pulsed ultrasonic signals into a body (e.g., patient) or volume. The ultrasonic signals may include, for example, one of more reference pulses, one or more pushing pulses (e.g., sheer-waves), and/or one or more tracking pulses. At least a portion of the pulsed ultrasonic signals are back-scattered from structures in and around an anatomical structure of interest and measured by the ultrasound probe 102. The anatomical structure of interest may be a brain, heart, bladder, kidney, liver, bone structure, vascular structure, organ, and/or the like. The ultrasound probe 102 may be connected wirelessly or with a cable to the portable host system 104. In one embodiment, the ultrasound probe 102 may be a universal probe, which integrates both a phased array transducer and a linear transducer into the same probe housing.

In various embodiments, the ultrasound probe 102 may include an analog front end (AFE) 220, shown in FIG. 2, which may include built-in electronics that enable the ultrasound probe 102 to transmit digital signals to the portable host system 104. The portable host system 104 then utilizes the digital signals to reconstruct an ultrasound image based on the information received from the ultrasound probe 102.

FIG. 2 is a schematic block diagram of the imaging system 100 shown in FIG. 1. In various embodiments, the ultrasound probe 102 includes a two-dimensional (2D) array 200 of elements. The ultrasound probe 102 may also be embodied as a 1.25D array, a 1.5D array, a 1.75D array, a 2D array, and/or the like. Optionally, the ultrasound probe 102 may be a stand-alone continuous wave (CW) probe with a single transmit element and a single receive element.

In various embodiments, the 2D array 200 may include a transmit group of elements 210 and a receive group of elements 212. Optionally, the ultrasound probe 102 may include a sub-aperture transmit beamformer 214 and a sub-aperture receive beamformer 218. The sub-aperture transmit beamformer 214 is configured to control a transmitter 216, which through sub-aperture transmit beamformer 214, drives the group of transmit elements 210 to emit, for example, CW ultrasonic transmit signals into a region of interest (e.g., human, animal, cavity, physical and/or anatomical structure, and/or the like) that includes the anatomical structure of interest (e.g., bladder, kidney, stomach, heart, uterus, liver, and/or the like). The transmitted CW ultrasonic signals are back-scattered from structures in and around the anatomical structure of interest, like blood cells, to produce echoes which return to the receive group of elements 212. The receive group of elements 212 convert the received echoes into analog signals as described in more detail below. The sub-aperture receive beamformer 218 is configured to partially beamforms the signals received from the receive group of elements 212 and then passes the partially beamformed signals to a receiver 228.

It may be noted in various embodiments, the ultrasound probe 102 may not include the sub-aperture transmit beamformer 214 and/or the sub-aperture receive beamformer 218. For example, the ultrasound probe 102 may include a controller circuit (not shown) having one or more processors, a central controller circuit (CPU), one or more microprocessors, or any other electronic component capable of processing inputted data according to specific logical instructions. The controller circuit may execute programmed instructions stored on a tangible and non-transitory computer readable medium (e.g., a memory, integrated memory of the controller circuit such as EEPROM, ROM, or RAM). Optionally, the controller circuit may perform one or more operations of the components of the ultrasound probe 102, such as a plurality of analog/digital (A/D) converters 222, the AFE 220, and/or the like. Additionally or alternatively, the operations of the sub-aperture transmit beamformer 214 may be performed by the transmitters 216. Optionally, the operations of the sub-aperture receiver beamformer 228 may be performed by the receiver 228.

In various embodiments, the receiver 228 may include the AFE 220. The AFE 220 may include for example, a plurality of demodulators 224 and the plurality of A/D converters 222. In operation, the complex demodulators 224 demodulate the RF signal to form IQ data pairs representative of the echo signals. The I and Q values of the beams represent in-phase and quadrature components of a magnitude of echo signals. More specifically, the complex demodulators 224 perform digital demodulation, and optionally filtering as described in more detail herein. The demodulated (or down-sampled) ultrasound data may then be converted to digital data using the A/D converters 222. The A/D converters 222 convert the analog outputs from the complex demodulators 224 to digital signals that are then transmitted to the portable host system 104 via a communication circuit 226.

Additionally or alternatively, the A/D converters 222 may be integrated and/or performed prior to the sub-aperture receive beamformer 216. For example, the echoes received from the elements 212 may be converted by the A/D converters 222 prior to being beamformed by the sub-aperture receive beamformer 216.

The communication circuit 226 may include hardware, such as a processor, controller circuit, or other logic based devices to transmit, detect and/or decode wireless data received by an antenna (not shown) of the communication circuit 226 based on a wireless protocol to and/or from the portable host system 104. For example, the wireless protocol may be Bluetooth, Bluetooth low energy, ZigBee, WiFi, and/or the like. Additionally or alternatively, the ultrasound probe 102 may be physically coupled to the portable host system 104 via a cable. For example, the digital information may be received by the portable host system 104 from the ultrasound probe 102 along the cable.

The ultrasound probe 102 may include a power source 221. The power source 221 is an electric power source, such as one or more batteries, a large capacitor, and/or the like. For example, the power source 221 may be a lithium ion battery, lead-acid battery, nickel cadmium battery, and/or the like. The power source 221 is conductively and/or electrically coupled to the components of the ultrasound probe 102. The power source 221 is configured to provide electrical power to the components of the ultrasound probe 102. Additionally or alternatively, the ultrasound probe 102 may receive electrical power from the portable host system 104. For example, the ultrasound probe 102 may be electrically coupled to the portable host system 104 via a wire (e.g., cable). The wire may be conductively coupled to a power source 236 of the portable host system 104, which delivers electrical power conducted along the wire from the power source 236 to the ultrasound prove 102.

The beamformers 214 and 218, and the complex demodulators 224 facilitate reducing the quantity of information that is transmitted from the ultrasound probe 102 to the portable host system 104. Accordingly, the quantity of information being processed by the portable host system 104 is reduced and ultrasound images of the patient may be generated, by the portable host system 104, in real-time as the information is being acquired from the ultrasound probe 102.

The portable host system 104 may include a controller circuit 202 operably coupled to the memory 204, the display 120, a user interface 238, and a communication circuit 230. The controller circuit 202 may include one or more processors. Additionally or alternatively, the controller circuit 202 may include a central processing unit (CPU), one or more microprocessors, a graphics processing unit (GPU), or any other electronic component capable of processing inputted data according to specific logical instructions. The controller circuit 202 may execute programmed instructions stored on a tangible and non-transitory computer readable medium (e.g., memory 204, integrated memory of the controller circuit 202 such as EEPROM, ROM, or RAM).

The portable host system 104 includes the power source 236. The power source 236 is an electric power source, such as one or more batteries, a large capacitor, and/or the like. For example, the power source 236 may be a lithium ion battery, lead-acid battery, nickel cadmium battery, and/or the like. The power source 236 is conductively and/or electrically coupled to the components of the portable host system 104. The power source 236 is configured to provide electrical power to the components of the portable host system 104.

The communication circuit 230 may include hardware, such as a processor, controller, or other logic based device to transmit, detect and/or decode wireless data received by an antenna (not shown) of the transceiver based on a wireless protocol (e.g., Bluetooth, Bluetooth low energy, ZigBee, WiFi, and/or the like). For example, the communication circuit 230 may transmit to and/or receive wireless data that includes ultrasound data from the communication circuit 226 of the ultrasound probe 102 along a bi-directional communication link. Additionally or alternatively, the communication circuit 230 may establish a bi-directional communication link with a remote server, such as a medical image server, a machine learning server, and/or the like.

The bi-directional communication link may be a wired (e.g., via a physical conductor) and/or wireless communication (e.g., utilizing radio frequency (RF)) link for exchanging data (e.g., data packets) between the ultrasound probe 102 and the portable host system 104. For example, the portable host system 104 may receive ultrasound data corresponding to first and second image data sets along the bi-directional communication link. The bi-directional communication link may be based on a standard communication protocol, such as Bluetooth, Ethernet, TCP/IP, WiFi, 802.11, a customized communication protocol, and/or the like.

In various embodiments, the portable host system 104 may include hardware components, including the controller circuit 202, that are integrated to form a single “System-On-Chip” (SOC). The SOC device may include multiple CPU cores and at least one GPU core. The SOC may be an integrated circuit (IC) such that all components of the SOC are on a single chip substrate (e.g., a single silicon die, a chip). For example, the SOC may have the memory 204, the controller circuit 202, the communication circuit 230 embedded on a single die contained within a single chip package (e.g., QFN, TQFP, SOIC, BGA, and/or the like).

The controller circuit 202 is operably coupled to the display 120 and the user interface 238. The user interface 238 controls operations of the controller circuit 202 and is configured to receive inputs from the user. The user interface 238 may include a keyboard, a mouse, a touchpad, one or more physical buttons (e.g., a button 122 shown in FIG. 1), and/or the like.

Optionally, the display 120 may be a touch screen display, which includes at least a portion of the user interface 238. For example, the display 120 may include a liquid crystal display, an organic light emitting diode display, and/or the like overlaid with a sensor substrate (not shown). The sensor substrate may include a transparent and/or optically transparent conducting surface, such as indium tin oxide (ITO), a metal mesh (e.g., a silver nano-tube mesh, and carbon match, a graph feed mesh), and/or the like. The sensor substrate may be configured as an array of electrically distinct rows and columns of electrodes that extend through a surface area of the display 120. The sensor substrate may be coupled to a touchscreen controller circuit (not shown). A touchscreen controller circuit may include hardware, such as a processor, a controller, or other logic-based devices and/or a combination of hardware and software which is used to determine a position on the display 120 activated and/or contacted by the user (e.g., finger(s) in contact with the display 120). In various embodiments, the touchscreen controller circuit may be a part of and/or integrated with the controller circuit 202 and/or a part of the display 120.

The touchscreen controller circuit may determine a user select position activated and/or contacted by the user by measuring a capacitance for each electrode (e.g., self-capacitance) of the sensor substrate. For example, a portion of the user interface 238 may correspond to a graphical user interface (GUI) generated by the controller circuit 202, which is shown on the display 120. The touch screen display can detect a presence of a touch from the operator on the display 120 and can also identify a location of the touch with respect to a surface area of the display 120. For example, the user may select one or more user interface icons of the GUI shown on the display by touching or making contact with the display 120. The touch may be applied by, for example, at least one of an individual's hand, glove, stylus, or the like.

The memory 204 includes parameters, algorithms, models, data values, and/or the like utilized by the controller circuit 202 to perform one or more operations described herein. The memory 204 may be a tangible and non-transitory computer readable medium such as flash memory, RAM, ROM, EEPROM, and/or the like. The memory 204 may include an image analysis algorithm configured to identify a rate of change of anatomical markers of one or more image data sets relative to each other. Additionally or alternatively, the image analysis algorithm may be received along one of the bi-directional communication links via the communication circuit 102 and stored in the memory 140. For example, the portable host system 104 may receive the image analysis algorithm from a remote server along the bi-directional communication link.

The image analysis algorithm may be based on a machine learning algorithm (e.g., convolutional neural network algorithms, deep learning algorithms, decision tree learning algorithms, and/or the like), a user pre-defined model, and/or the like. The image analysis algorithm is configured to identify anatomical markers of an anatomical structure of interest (e.g., brain, heart, bladder, kidney, liver, bone structure, vascular structure, organ, and/or the like) within one or more image data sets. The anatomical markers may represent structures (e.g., chambers, anatomical boundaries, tracts, apertures, and/or the like), landmarks (e.g., apex), and/or features of the anatomical structure of interest.

For example, the controller circuit 202 may be configured to receive a plurality of image data sets of an anatomical structure of interest. The image data sets may represent ultrasound data acquired by the ultrasound probe 102. The controller circuit 202 (e.g., by executing the image analysis algorithm) is configured to identify one or more anatomical markers of the anatomical structure and assign the anatomical markers a coordinate within the plurality of image data sets. The coordinate may be based on an anatomical coordinate system of the anatomical structure of interest defined by the image analysis algorithm. Additionally or alternatively, the coordinate may be based on a position relative to the corresponding image data set. The controller circuit 202 may calculate a change in positions of the coordinates of the one or more anatomical markers between adjacent and/or two temporally different image data sets. Additionally or alternatively, the controller circuit 202 may determine a rate of change in positions. For example, the controller circuit 202 may determine a change in position relative to time between acquisition of the adjacent and/or two temporally different image data sets to calculate a rate of change in position. Optionally, the rate of change may represent a velocity.

The image analysis algorithm may be defined by a machine learning algorithm (e.g., convolutional neural network algorithms, deep learning algorithms, decision tree learning algorithms, and/or the like) utilizing a series of training images of the anatomical structure of interest. The anatomical markers may be identified by the machine learning algorithm based on features of the one or more anatomical structures (e.g., boundaries, thickness, and/or the like). The features may represent high level features of the pixels and/or voxels of the training images such as a histogram orient gradients, blob features, covariance features, binary pattern features, and/or the like. Optionally, the machine learning algorithm may define the image analysis algorithm by automatically building a statistical model and/or a database of true positives and true negatives corresponding to each anatomical marker identified based on the features from the training images, a classification model, supervised modeling, and/or the like.

For example, the image analysis algorithm may be configured and/or designed based on a plurality of training medical images. The plurality of training medical images may be grouped into different anatomical marker sets. Additionally or alternatively, the training medical images within each set may represent different orientations and/or views of the one or more anatomical markers. For example, a set of the training medical images may include over 50,000 images. For example, a set of the training medical images may include one or more different views corresponding to the heart (e.g., a possible anatomical structure of interest). In another example, a second set of the training images may include one or more different views corresponding to the brain (e.g., a possible anatomical structure of interest).

Additionally or alternatively, the image analysis algorithm may be defined based on a supervised learning method to identify the anatomical markers within the one or more image data sets. For example, a user (e.g., skilled medical practitioner) may manually label the one or more anatomical markers within the plurality of training medical images utilizing the user interface 238. The manually labeled medical images may be used to build a statistical model and/or a database of true positives and true negatives corresponding to each anatomical marker of the anatomical structure of interest defining the image analysis algorithm.

The image analysis algorithm may be defined to identify the one or more anatomical markers utilizing a classification model (e.g., random forest classifier). For example, the image analysis algorithm may be configured to identify the one or more anatomical markers based on a pixel level classifier model to label and/or assign each pixel of the medical image into a plurality of categories or classes (e.g., muscle, fat, background anatomy, anatomical structure of interest, chambers). The controller circuit 202 executing the classification model may determine the classes from a feature space of the pixels based from the various intensities and spatial positions of pixels within the image data set. The controller circuit 202 executing the image analysis algorithm may continually select a pixel of the first and second image data sets, and compare characteristics of the select pixel to feature vectors. For example, the controller circuit 202 may compare an intensity or brightness of the select pixel to feature vectors of the classification model. In another example, the controller circuit 202 may determine a variance kurtosis, skewness, or spatial distribution characteristic of the select pixel by comparing the intensity of the select pixel with adjacent and/or proximate pixels around the select pixel.

A number of characteristics of the select pixel, that are compared by the controller circuit 202, may be based on the feature sets included in the feature vectors. Each feature vector may be an n-dimensional vector that includes three or more features of pixels (e.g., mean, variance, kurtosis, skewness, spatial distribution) corresponding to a class (e.g., a background anatomy, muscle tissue, fat, the bladder) of pixels of anatomical markers within the first and second image data sets. The feature vectors of the classification model may be generated and/or defined by the controller circuit 202 based on a plurality of training medical images. For example, the controller circuit 202 may select pixel blocks from one hundred reference training medical images. The select pixel blocks may have a length of five pixels and a width of five pixels. For example, a plurality of pixels within each select pixel block may represent and/or correspond to one of the classes, such as tissue of the anatomical structure of interest. Based on the plurality of pixels within the select pixel blocks, the controller circuit 202 may generate and/or define a feature vector. The controller circuit 202 may determine feature sets for each pixel within the plurality of pixels of a select pixel block or more than one select pixel block corresponding to the same class. One of the feature sets may be based on an intensity histogram of the training medical images. For example, the controller circuit 202 may calculate a mean intensity of the plurality of pixels, a variance of the plurality of pixel intensities, a kurtosis or shape of intensity distribution of the plurality of pixels, a skewness of the plurality of pixels, and/or the like.

Additionally, one of the feature sets may correspond to a position or spatial feature of the pixels within the select pixel block. A spatial position with respect to a position within the reference image (e.g., central location) and a depth with respect to an acquisition depth within the patient. The controller circuit 202 may perform a k-means clustering and/or random forest classification on the feature sets to define feature values that correspond to the class of the select pixel blocks. The controller circuit 202 may define a feature vector corresponding to the class based on the feature values to the classification model. The controller circuit 202 may assign a class to the select pixel based on a corresponding feature vector. When the select pixel is assigned a class, the controller circuit 202 may repeat the classification model to the remaining pixels of the first and second image data sets to identify the anatomical markers.

It may be noted that the machine learning algorithms utilized to define the image analysis algorithm are examples, additional methods are available for a person of ordinary skill in the art.

The controller circuit 202 may execute programmed instructions stored in the memory 204 to instruct the ultrasound probe 102 to start acquiring ultrasound data. For example, the controller circuit 202 may execute the programmed instructions based on a user input received from the user interface 238. During acquisition, the ultrasound probe 102 may be manually positioned and/or moved by the clinician to a region of interest. The ultrasound probe 102 emits pulsed ultrasound signals from the transducer array 106 into the region of interest at a set rate, such as a pulse repetition time (PRT). The PRT may be based on the imaging depth of the anatomical structure of interest. The ultrasonic signals may include, for example, one or more reference pulses, one or more pushing pulses (e.g., shear-waves), and/or one or more pulsed wave Doppler pulses. At least a portion of the pulsed ultrasonic signals back-scatter from the anatomical structure of interest to produce echoes. The echoes are delayed in time and/or frequency according to a depth or movement, and are received by the transducer elements 212 within the transducer array 106. An amount of time for the ultrasound signals to traverse to the anatomical structure of interest and back-scatter from the anatomical structure of interest to the transducer array 106 may represent a minimum PRT. A plurality of the ultrasonic signals corresponding to different minimum PRT sets that may be used for generating an ultrasound image, for generating and/or tracking shear-waves, for measuring changes in position or velocity within the anatomic structure, differences in compression displacement of the tissue (e.g., strain), and/or for therapy, among other uses. For example, the ultrasound probe 102 may deliver low energy pulses during imaging and tracking, medium to high energy pulses to generate shear-waves, and high energy pulses during therapy.

Based on the ultrasound data received by the transducer array 106, the controller circuit 202 generates one or more image data sets. Each of the image data sets are temporally different, such as corresponding to ultrasound data acquired at different points in time and/or minimum PRT sets. The image data sets may be generated by the controller circuit 202 based on programmed instructions stored in the memory 204 (FIG. 2). For example, the programmed instructions may include algorithms for beamforming as well as subsequent signal and image processing steps utilized to process (e.g., an RF processor 232) and display the image data sets based on the ultrasound data received from the ultrasound probe 102.

The controller circuit 202 may execute the beamforming algorithm stored in the memory 204 to perform additional or final beamforming to the digital ultrasound information received from the ultrasound probe 102, and outputs a radio frequency (RF) signal. Additionally or alternatively, the portable host system 104 may include a receive beamformer (not shown), which receives the digital ultrasound information and performs the additional or final beamforming. The RF signal is then provided to an RF processor 232, which is configured to process the RF signal. The RF processor 232 may include a complex demodulator 234 that demodulates the RF signal to form IQ data pairs representative of the echo signals, and one or more processors. The RF or IQ signal data may then be provided directly to the memory 204 for storage (e.g., temporary storage). Optionally, the output of the RF processors 232 may be passed directly to the controller circuit 202. Additionally or alternatively, the RF processor 232 may be integrated with the controller circuit 202 corresponding to programmed instructions of the ultrasound imaging application stored in the memory 204.

The controller circuit 202 may further process the output of the RF processor 232 and to generate the one or more image data sets for display on the display 120. In operation, the controller circuit 202 is configured to perform one or more processing operations according to a plurality of selectable ultrasound modalities on the acquired ultrasound data.

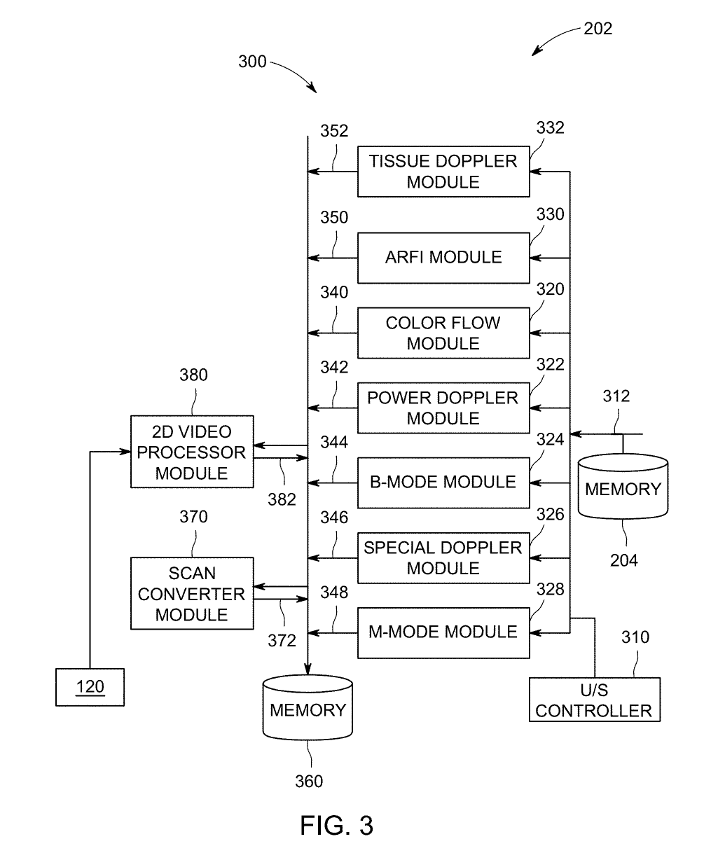

FIG. 3 illustrates an exemplary block diagram of an embodiment of the controller circuit 202 of the portable host system 104. The controller circuit 202 may include an ultrasound processor circuit 300, which is illustrated conceptually as a collection of sub-modules corresponding to operations that may be performed by the controller circuit 202 when executing programmed instructions for acquiring the image data sets. Optionally, the one or more sub-modules may be implemented utilizing any combination of dedicated hardware boards, DSPs, processors, and/or the like of the portable host system 104. Additionally or alternatively, the sub-modules of FIG. 3 may be implemented utilizing one or more processors, with the functional operations distributed between the processors, for example also including a Graphics Processor Unit (GPU). As a further option, the sub-modules of FIG. 3 may be implemented utilizing a hybrid configuration in which certain modular functions are performed utilizing dedicated hardware, while the remaining modular functions are performed utilizing a processor. The sub-modules also may be implemented as software modules within a processing unit.

The operations of the sub-modules illustrated in FIG. 3 may be controlled by a local ultrasound controller 310 or by the controller circuit 202. The controller circuit 202 may receive ultrasound data 312 in one of several forms. In the exemplary embodiment of FIG. 2, the received ultrasound data 312 constitutes IQ data pairs representing the real and imaginary components associated with each data sample. The IQ data pairs are provided to one or more of a color-flow sub-module 320, a power Doppler sub-module 322, a B-mode sub-module 324, a spectral Doppler sub-module 326 and an M-mode sub-module 328. Optionally, other sub-modules may be included such as an Acoustic Radiation Force Impulse (ARFI) sub-module 330 and a Tissue Doppler (TDE) sub-module 332, among others.

Each of sub-modules 320-332 are configured to process the IQ data pairs in a corresponding manner to generate color-flow data 340, power Doppler data 342, B-mode data 344, spectral Doppler data 346, M-mode data 348, ARFI data 350, and tissue Doppler data 352, all of which may be stored in a memory 360 and/or the memory 204 temporarily before subsequent processing. For example, the B-mode sub-module 324 may generate B-mode data 344 including a plurality of B-mode ultrasound images corresponding to one or more image data sets. The data 340-352 may be stored in the memory 360, 204 for example, as sets of vector data values, where each set defines an individual ultrasound image frame. The vector data values are generally organized based on the polar coordinate system.

A scan converter sub-module 370 accesses and obtains from the memory 360, 204 the vector data values associated with an image frame and converts the set of vector data values to Cartesian coordinates to generate an ultrasound image frames 372 (e.g., one of the image data sets) formatted for display on the display 120. The ultrasound image frames 372 generated by the scan converter module 370 may be provided back to the memory 360, 204 for subsequent processing.

Once the scan converter sub-module 370 generates the ultrasound image frames 372 associated with, for example, the B-mode ultrasound image data, and/or the like, the ultrasound image frames 372 may be restored in the memory 360 or communicated over a bus to a database (not shown), the memory 360, 204, and/or to other processors. The scan converted data may be converted into an X, Y format for display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the controller circuit 202 instructs the display 120 (shown in FIG. 2) to display the image data sets.

A video processor sub-module 380 may combine one or more of the frames generated from the different types of ultrasound information. For example, the video processor sub-module 380 may combine different image frames by mapping one type of data to a grey map and mapping the other type of data to a color map for video display. In the final displayed image data set, color pixel data may be superimposed on the grey scale pixel data to form a single multi-mode image frame 382 (e.g., functional image) that is again stored in the memory 360, 204 or communicated over the bus. Successive frames of a plurality of image data sets may be stored as a cine loop in the memory 360, 204. The cine loop represents a first in, first out circular image buffer to capture image data that is displayed to the user.

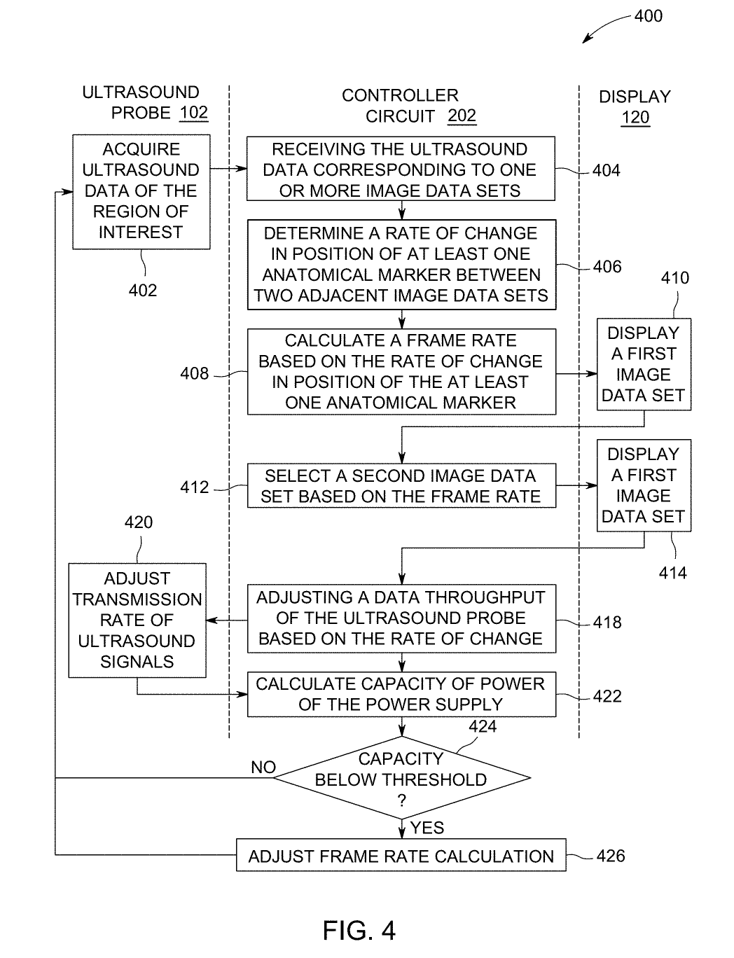

In connection with FIG. 4, the controller circuit 202 may identify anatomical markers within the image data sets stored in the memory 360, 204, and determine a rate of change in position of the anatomical markers between the one or more image date sets. Based on the rate of change, the controller circuit 202 may calculate a frame rate of the image data sets. The controller circuit 202 may instruct the 2D video processor sub-module 380 to display the image data sets successively at the frame rate to be displayed on the display 120.

FIG. 4 illustrates a swim lane diagram illustrating a method of using a mobile ultrasound imaging system to automatically adjust a frame rate, in accordance with various embodiments described herein. The method 400, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 400 may be used as one or more algorithms or applications to direct hardware to perform one or more operations described herein. It should be noted, other methods may be used, in accordance with embodiments herein.

Beginning at 402, the ultrasound probe 102 acquires ultrasound data of the region of interest (ROI). During a scan of the ROI, which includes the anatomical structure of interest, the ultrasound probe 120 may emit ultrasound signals from the transducer array 106 at a set rate. The ultrasound data is transmitted from the communication circuit 226 along a bi-directional communication link to the communication circuit 230.

At 404, the controller circuit 202 is configured to receive the ultrasound data corresponding to one or more image data sets. For example, the controller circuit 202 may receive the ultrasound data from the communication circuit 230 operatively coupled to the controller circuit

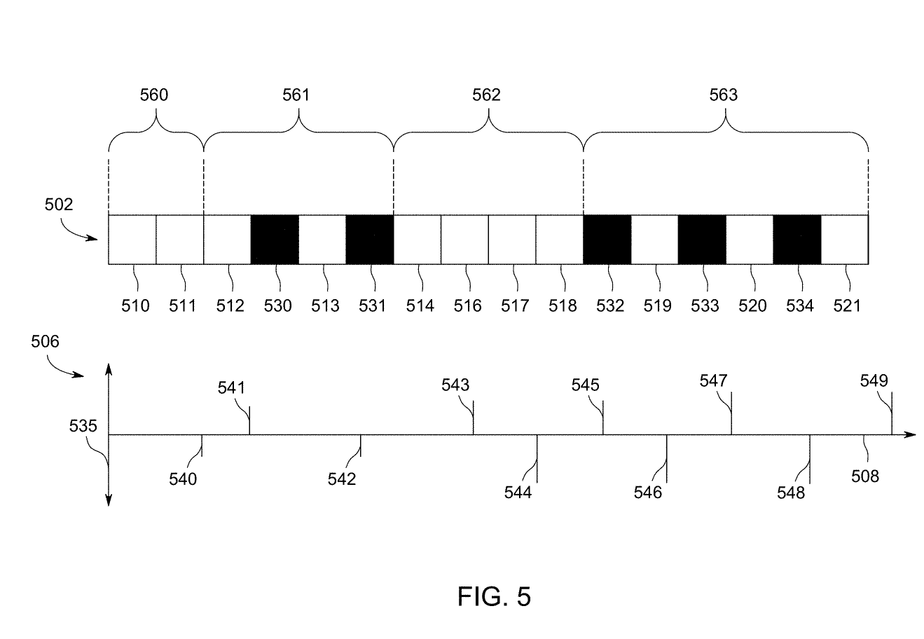

At 406, the controller circuit 202 is configured to calculate a rate of change in position of at least one anatomical marker between two adjacent image data sets. FIG. 5 illustrates an embodiment of a rate of change in position of at least one anatomical marker and acquisition frame rates 560-563. The ultrasound data 502 received by the controller circuit 202 along the bi-directional communication link is shown along a horizontal axis 508 representing time. The ultrasound data 502 is subdivided into a plurality of image data sets 510-521. For example, the image data sets 510-521 may represent an amount of ultrasound data that may be utilized by the controller circuit 202 to generate ultrasound images representative of each image data set 510-521. Optionally, the image data sets 510-521 over time may represent a data throughput of the ultrasound probe 102 along the bi-directional communication link. For example, the number of the image data sets 510-521 overtime may represent an amount of data being transmitted by the ultrasound probe 102 along the bi-directional communication link. It may be noted that the image data sets 510-521 are acquired over time, and are temporally different. The image data sets 510-521 may correspond to ultrasound data acquisitions by the ultrasound probe 102. A number of the image data sets 510-521 over time define different acquisition frame rates 560-563. For example, the image data sets 510-511 may define a first acquisition frame rate 560, the image data sets 512-513 may define a second acquisition frame rate 561, the image data sets 514-518 may define a third acquisition frame rate 562, and the image data sets 519-521 may define a fourth acquisition frame rate 563. The acquisition frame rates 560-563 may be based on a rate of change in position of the at least one anatomical marker.

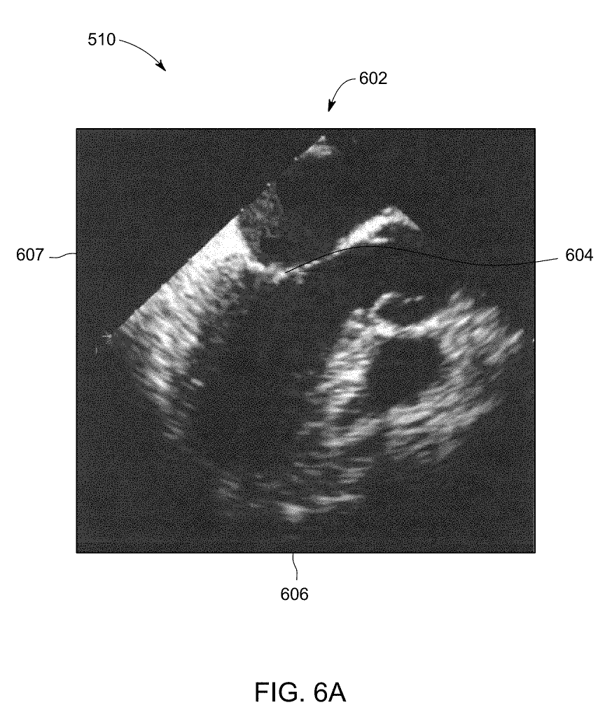

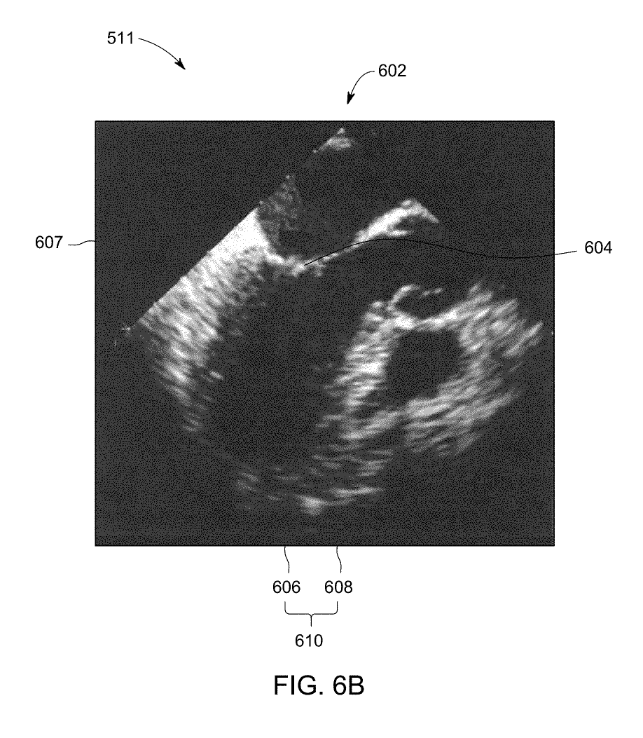

The controller circuit 202 may determine a rate of change in position of the at least one anatomical marker when the image data sets 510-521. For example, the controller circuit 202 may execute the image analysis algorithm when each image data set 510-521 is received. In connection with FIGS. 6A-B, the controller circuit 202 may determine a rate of change in position of at least one anatomical marker 604 between two adjacent and/or successive image data sets 510, 511. The adjacent image data sets 510, 511 may represent two image data sets 510, 511 received by the controller circuit 202 successively in time.

FIGS. 6A-B illustrate image data sets 510, 511 of an embodiment of an anatomical structure 602 of interest at different points in time. For example, the anatomical structure 602 of interest is shown as a heart. The controller circuit 202 may execute the image analysis algorithm when the image data set 510 is received by the controller circuit 202. The controller circuit 202 (e.g., by executing the image analysis algorithm) may identify a position of at least one anatomical marker 604. For example, the anatomical marker 604 identified may represent a valve of the anatomical structure 602 of interest. It may be noted that in various embodiments, the controller circuit 202 may identify additional anatomical markers, such as a plurality of valves, ventricular boundaries, centroids of one or more cavities, and/or the like. The controller circuit 202 may identify a position of the anatomical marker 604 at a coordinate at 606 along an X-axis and at 607 along a Y-axis.

When the image data set 511 is received by the controller circuit 202, the controller circuit 202 may execute the image analysis algorithm to identify the at least one anatomical marker 604, such as the same anatomical marker(s) 604 identified in the image data set 510. The controller circuit 202 may identify a position of the anatomical marker 604 of the image data set 511 at a coordinate at 608 along an X-axis and at 607 along a Y-axis. The controller circuit 202 may calculate a difference in position and/or distance 610 traversed by the anatomical marker 604 between image data sets 510, 511 based on changes in coordinates. Based on the distance, the controller circuit 202 may determine a rate of change in position. For example, the controller circuit 202 may determine a time between receiving the image data sets 510, 511, corresponding to a minimum PRT. Optionally, the controller circuit 202 may determine a velocity of the rate change based on the time between the image data set 510, 511 and the distance traversed by the anatomical marker 604.

At 408, the controller circuit 202 may be configured to determine an acquisition frame rate based on the rate 540-549 of change in position of the at least one anatomical marker. The graphical illustration 506 may represent the rates 540-549 of changes in position of the at least one anatomical marker 604 between adjacent image data sets 540-549. The controller circuit 202 may compare the rate 540 to a rate database stored in the memory 204 to determine the acquisition frame rates 560-563. For example, the rate database may correspond to a collection of different rates with corresponding acquisition frame rates 560-563. The controller circuit 202 may identify the rate within the database to determine the acquisition frame rates 560-5634 for the ultrasound probe 102. For example, based on the rate 541 the controller circuit 202 may determine the first acquisition frame rate 561. The controller circuit 202 may adjust the acquisition frame rates 560-563 by adjusting the PRT and/or adjusting the frame time based on Equation (1) shown below.

$$Framerate = \frac{1}{[ {N}_{beams} * ( {PRT}_{min} + {delta}_{PRT} ) + {delta}_{T\_frame} ]} \qquad Equation ( 1 )$$

Equation (1) may define the acquisition frame rates 560-563. The variable N_{beams} may represent a number of ultrasound signals emitted from the transducer array 106 to acquire the image data sets 510-521. The variable PRTₘᵢₙ may represent an amount of time for the ultrasound signals to traverse a depth within the patient to the anatomical structure of interest and back-scatter from the anatomical structure of interest to the transducer array 106. The variables delta_{PRT} and delta_{T}_{_}_{frame} may correspond to methods for the controller circuit 202 to adjust the acquisition frame rates 560-563. Each of the variables, delta_{PRT} and delta_{T}_{_}_{frame}, may represent additional time the controller circuit 202 may add to adjust a length of the acquisition frame rates 560-563. The variable delta_{PRT} is configured to add additional time to the PRTₘᵢₙ. The variable delta_{T}_{_}_{frame}, is configured to add a pause and/or time between image data sets 510-521 acquired by the ultrasound probe 102.

For example, the first acquisition frame rate 560 may correspond to the variables delta_{PRT} and delta_{T}_{_}_{frame} equal to and/or at zero (e.g., the acquisition frame rate 560 may be at PRTₘᵢₙ). Based on the rate 540, the controller circuit 202 may determine that the first acquisition frame rate 560 can be adjusted and/or reduced with respect to the rate database stored in the memory 204. The adjustment to the second acquisition frame rate 561 may be performed by the controller circuit 202 by increasing at least one of the variables delta_{PRT} and delta_{T}_{_}_{frame}. For example, the adjustment in the frame rate may be represented as the data sets 530-531. The data sets 530-531 may be interposed between adjacent image data sets 512-513, respectively. The data sets 530-531 correspond to an amount of time where no ultrasound signals are emitted by the transducer array 106, no ultrasound data is transmitted and/or received along the bi-directional communication link, and/or ultrasound data is processed by the portable host system 104 (e.g., the controller circuit 202, and/or the like). During the data sets 530-531 components (e.g., the communication circuit 226, the transmitter 216, the receiver 228, the AFE 220, and/or the like) of the ultrasound probe 102 may be configured to be disabled and/or not consume power from the battery 221 and/or 236 (e.g., when connected via a cable to the portable host system 104). Additionally or alternatively, during the data sets 530-531 the components (e.g., the communication circuit 230, the RF processors 232, and/or the like) may be configured to be disabled and/or not consumer power from the battery 236. As the components of the ultrasound probe 102 and/or the portable host system 104 are disabled during the data sets 530-531, there is less processing demands for the system 100 reducing power consumption. Additionally, the data sets 530-531 are configured to increase the second acquisition frame rate 561 relative to the first acquisition frame rate 560. A length of time for the data sets 530-531 may correspond to an amount of time of the variables delta_{PRT} and delta_{T}_{_}_{frame}.

The dynamic adjustment to the first acquisition frame rate 560 to the second acquisition frame rate 561 based on the rates 540-549 may be configured to reduce the power consumption of the portable host system 104. For example, the reduction of a rate of a number of image data sets 512-513, relative to the image data sets 510-511, received by the portable host system 104 reduces an amount of processing required by the controller circuit 202 to generate ultrasound images to be displayed on the display 120. Further, the reduction in the power consumption reduces an amount of heat generated by the components (e.g., the controller circuit 202) of the portable host system 104. Additionally or alternatively, the change in the acquisition frame rate 561 reduces a throughput along the bi-directional communication link between the portable host system 104 and the ultrasound probe 102. The reduction in throughput is based on a reduction in an amount of image data sets 512-513 transmitted along the bi-directional communication link over time. The adjustment in the throughput further reduces the power consumption of the ultrasound probe 102. For example, the reduction in the acquisition frame rate from 560 to 561 reduces a rate of the ultrasound signals being transmitted by the transducer array 106.

At 410, the display 120 is configured to display a first image data set. For example, the controller circuit 202 may select the image data set 512 as the first image data set acquired at the acquisition frame rate 561. When the controller circuit 202 selects the image data set 512, the controller circuit 202 is configured to generate an ultrasound image based on the image data set 512 and is sent to the display 120.

At 412, the controller circuit 202 is configured to select a second image data set based on the acquisition frame rate 561. For example, the controller circuit 202 may select the image data set 513 that was acquired within the acquisition frame rate 561 as the second image data set shown on the display 120. When the controller circuit 202 selects the image data set 513, the ultrasound image is sent to the display 120.

At 414, the display 120 is configured to display the second image data set. For example, when the controller circuit 202 selects the image data set 513, the ultrasound image corresponding to the image data set 513 is sent to the display 120.

It may be noted that the controller circuit 202 may continually determine changes in position of the anatomical marker 604 between the adjacent image data sets 510-521 to dynamically change the acquisition frame rates 560-563. Additionally, the controller circuit 202 may instruct the display 120 to display the image data sets 510-521 acquired at the acquisition frame rates 560-563.

For example, the controller circuit 202 may calculate the rate 543 based on a change in position of the anatomical marker 604 between the image data sets 513 and 514. The controller circuit 202 may compare the rate with the frame rate database in the memory 204 and adjust the second acquisition frame rate 561 to the third acquisition frame rate 562. For example, the rate 543 may be higher than the rate 542 such that the controller circuit 202 is configured to reduce the acquisition frame rate. For example, based on the movement of the anatomical marker 604 between the image date sets 513-514 the second acquisition frame rate 561 is increased to reduce potential loss of relevant clinical information during the scan to the third acquisition frame rate 562. Based on the increase in the acquisition frame rate 562 corresponding to the rate database, the controller circuit 202 may be configured to reduce the variables delta_{PRT} and delta_{T}_{_}_{frame} to zero. For example, the third acquisition frame rate 562 may be approximately at PRTₘᵢₙ. It may be noted that due to the reduced third acquisition frame rate 562 relative to the second acquisition frame rate 561, a data throughput of the ultrasound probe 102 is adjusted.

Returning to FIG. 4, at 418, the controller circuit 202 is configured to adjust a data throughput of the ultrasound probe 102 based on the rate of change. For example, the throughput along the bi-directional communication link may be based on a number of image data sets 510-521 transmitted over time. The throughput is based on the acquisition frame rates 560-563. For example, an amount of data transmitted along the bi-directional communication link for the third acquisition frame rate 562 is increased relative to the second acquisition frame rate 561. Due to the removal of data sets 530-531, an increased number of image data sets 514-518 over time are transmitted along the bi-directional communication link from the ultrasound probe 102 to the portable host system 102. For example, the second acquisition frame rate 561 includes data sets 530-531 of which no ultrasound data (e.g., image data sets) are being transmitted along the bi-directional communication link. Alternatively, the third acquisition frame rate 562 does not include the data sets 530-534. Due to the increased number of image data sets 514-518 over time, the power consumption of the ultrasound probe 102 and the portable host system 104 is increased relative to the second acquisition frame rate 561. For example, the controller circuit 202 must process more data (e.g., image data sets 514-518) based on the change in the acquisition frame rate 562. Additionally, due to the increase in power consumption of the portable host system 104, more the components (e.g., the controller circuit 202) may generate more heat based on the increased in processing of the image data sets 514-518 over time.

The controller circuit 202 may transmit an instruction along the bi-directional communication link to the ultrasound probe 102 via the communication circuits 226, 230 to adjust the acquisition frame rate. For example, the controller circuit 202 may instruct the ultrasound probe 102 to adjust the acquisition frame rate from the second acquisition frame rate 561 to the third acquisition frame rate 562 along the bi-directional communication link.

At 420, the ultrasound probe 102 is configured to adjust a transmission rate of the ultrasound signals based on the acquisition frame rate. For example, based on the instructions received by the controller circuit 202, the ultrasound probe 102 may increase the transmission rate of the ultrasound signals corresponding to the third acquisition frame rate 562 relative to the second acquisition frame rate 561. During the second acquisition frame rate 561, during the data sets 530-531 ultrasound signals were not emitted by the transducer array 106. Conversely, during the third acquisition frame rate 562 the ultrasound probe 102 continually transmits ultrasound signals corresponding to the image data sets 514-518, increasing the transmission rate.

In another example, the controller circuit 202 may instruct the ultrasound probe 102 to reduce the throughput transmitted along the controller circuit 202. For example, the controller circuit 202 may calculate the rate 546 based on a change in position of the anatomical marker 604 between the image data sets 517 and 518. The controller circuit 202 may compare the rate with the frame rate database in the memory 204 and adjust the third acquisition frame rate 562 to the fourth acquisition frame rate 563. For example, the rate 546 may be lower than the rate 545 such that the controller circuit 202 is configured to reduce the acquisition frame rate. The rate 546 may correspond to a reduction in potential loss of relevant clinical information during the scan to the fourth acquisition frame rate 563.

Based on the decrease in the acquisition frame rate 563 corresponding to the rate database, the controller circuit 202 may be configured to increase at least one of the variables delta_{PRT} and delta_{T}_{_}_{frame} to increase the acquisition frame rate to form the fourth acquisition frame rate 563. For example, based on the increase in at least one of the variables delta_{PRT} and delta_{T}_{_}_{frame} the data sets 532-534 are formed within the fourth acquisition frame rate 563. The data sets 532-534 are interposed between adjacent image data sets 519-521, respectively. The data sets 532-534 correspond to time where no ultrasound data is transmitted along the bi-directional communication link and/or processed by the portable host system 104 (e.g., the controller circuit 202, and/or the like). It may be noted that due to the increased fourth acquisition frame rate 563 relative to the third acquisition frame rate 562, a data throughput of the ultrasound probe 102 is adjusted. For example, an amount of data transmitted along the bi-directional communication link is decreased relative to the third acquisition frame rate 562. Due to the fourth acquisition frame rate 563, a decreased number of image data sets 519-521 over time are transmitted along the bi-directional communication link from the ultrasound probe 102 to the portable host system 102. Due to the decrease in the number of image data sets 519-521, the power consumption of the ultrasound probe 102 and the portable host system 104 is decreased relative to the third acquisition frame rate 562. For example, the controller circuit 202 must process less data (e.g., image data sets 519-521) based on the change in the acquisition frame rate 563. Additionally, due to the decrease in power consumption of the portable host system 104, the components (e.g., the controller circuit 202) may generate less heat based on the decrease in processing of the image data sets 519-521 over time.

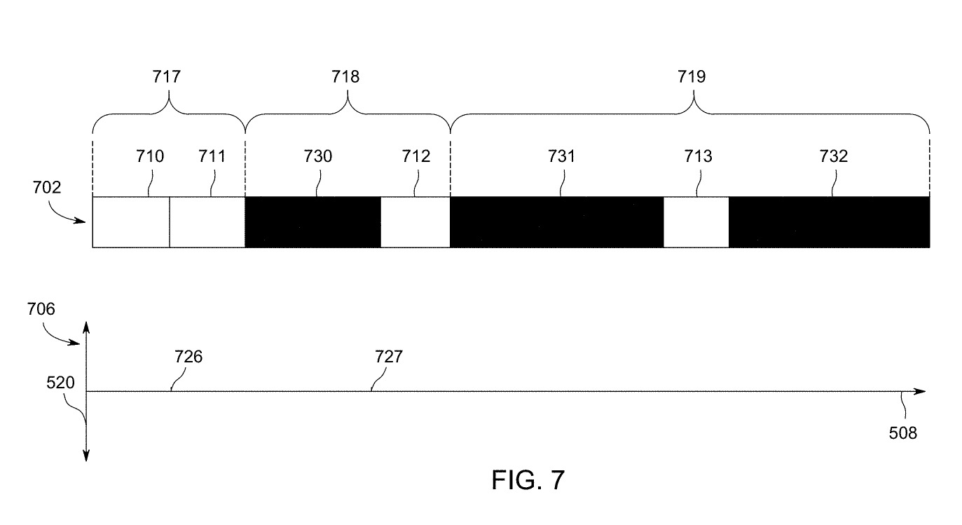

Optionally, a size of the data sets 530-534 may be greater than shown in FIG. 5. FIG. 7 illustrates an embodiment of a rate 726-727 of change in position of at least one anatomical marker and the acquisition frame rates 717-719. The anatomical structure of interest shown in FIG. 7 may be a static and/or stationary. The static and/or stationary anatomical structure may receive limited and/or low amount of nerve signals. For example, the anatomical structure of interest may be a bladder, kidney, liver and/or the like. Ultrasound data 702 received by the controller circuit 202 along the bi-directional communication link is shown along the horizontal axis 508 representing time. The ultrasound data 702 includes image data sets 710-713. The ultrasound data 702 includes data sets 730-732. The data sets 730-732 are similar to and/or the same as the data sets 530-534. For example, during the data sets 730-732 no ultrasound signals are emitted by the transducer array 106, no ultrasound data is transmitted and/or received along the bi-directional communication link, and/or ultrasound data is processed by the portable host system 104 (e.g., the controller circuit 202, and/or the like).

The graphical illustration 706 may represent the rates 726-727 of changes in position of the at least one anatomical marker of the image data sets 710-713. The controller circuit 202 may compare the rates 726-727 to a rate database stored in the memory 204 to determine the acquisition frame rate 717-719. Based on the stationary anatomical structure of interest, the rates 726-727 are lower, relative to the rates 540-549, resulting in the lower acquisition frame rates 717-719, relative to the acquisition frame rates 560-563.

For example, the controller circuit 202 may calculate the rate 726 based on a change in position of the at least one anatomical marker between the image data sets 710 and 711. The controller circuit 202 may compare the rate with the frame rate database in the memory 204 and adjust a first acquisition frame rate 717 to a second acquisition frame rate 718. The second acquisition frame rate 718 include a date set 730 based on the adjustment to at least one of the variables delta_{PRT} and delta_{T}_{_}_{frame} by the controller circuit 202. The data sets 730 may be interposed between adjacent image data sets 711 and 712.

As the rate 727 decreases further, the controller circuit 202 may extend a length of the data sets 731-732. For example, the controller circuit 202 may calculate the rate 727 based on a change in position of the at least one anatomical marker between the image data sets 711 and 712. The rate 727 may be approximately within a predetermined non-zero threshold (e.g., within a set percentage of less than 1 percent, a margin, and/or the like) and/or at zero. The controller circuit 202 may compare the rate with the rate database in the memory 204 and adjust the second acquisition frame rate 718 to the third acquisition frame rate 719. For example, the third acquisition frame rate 719 may represent a static acquisition frame rate. The static acquisition frame rate may correspond to a maximum increase of the the variables delta_{PRT} and delta_{T}_{_}_{frame} by the controller circuit 202. During the static acquisition frame rate the portable host system 104 and the ultrasound probe 102 may correspond to a lowest power consumption relative to alternative acquisition frame rates.

For example, the reduction of a rate of a number of image data sets 713, relative to a rate of the image data sets 710-712, received by the portable host system 104 reduces an amount of processing required by the controller circuit 202 to generate ultrasound images to be displayed on the display 120. Further, the reduction in the power consumption reduces an amount of heat generated by the components (e.g., the controller circuit 202) of the portable host system 104 and the components (e.g., the transmitter 216, receiver 228, the AFE 220) of the ultrasound probe 102. Additionally, the third acquisition frame rate 719 reduces a throughput along the bi-directional communication link between the portable host system 104 and the ultrasound probe 102. The reduction in throughput is based on a reduction in an amount of image data sets 713 transmitted along the bi-directional communication link over time relative to the alternative acquisition frame rates 560-563, 717-718. The adjustment in the throughput further reduces the power consumption of the ultrasound probe 102. For example, the reduction corresponding to the third acquisition frame rate 719 reduces a rate of the ultrasound signals being transmitted by the transducer array 106.

Returning to FIG. 4, at 422, the controller circuit 202 is configured to calculate a capacity of power of the power supply 236. For example, the controller circuit 202 may measure a discharged rate of the power supply 236. The discharged rate may represent the current discharged by the power supply 236 and/or demanded by the components of the portable host system 104 relative to the draw current of the power supply 236. Additionally or alternatively, the controller circuit 202 may measure a voltage potential across the power supply 236 to determine the capacity of charge remaining.

At 424, the controller circuit 202 may be configured to determine whether the capacity of the power supply 236 is below the threshold. For example, the threshold may be a predetermined non-zero threshold stored in the memory 204. The threshold may represent an amount of capacity of the power supply 236 to continue an ultrasound exam for a period of time. Optionally, the threshold may represent an amount of capacity of the power supply 236 to have the acquisition frame rate at set rate (e.g., PRT_{MIN}) for a period of time.

If the capacity is at and/or below the threshold, then at 426, the controller circuit 202 adjusts the acquisition frame rate determination. For example, the controller circuit 202 may decrease the acquisition frame rate by a set predetermined percentage, predetermined margin, and/or the like. For example, the controller circuit 202 may increase a value of at least one of the variables delta_{PRT} and delta_{T}_{_}_{frame}. Optionally, the capacity below the threshold may correspond to a lower power mode designated by the controller circuit 202. For example, during the low power mode the controller circuit 202 may adjust (e.g., lower) the acquisition frame rate, decrease the data throughput along the bi-directional communication link, and/or the like. Additionally or alternatively, the controller circuit 202 may display a graphical icon, textual information, and/or the like on a GUI shown on the display 120.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem,” “circuit,” “controller circuit,” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term computer,” “subsystem,” “circuit,” “controller circuit,” or “module”.

The “computer,” “subsystem,” “circuit,” “controller circuit,” or “module” executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer,” “subsystem,” “circuit,” “controller circuit,” or “module” to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method comprising:
receiving ultrasound data representing image data sets of an anatomical structure of interest, wherein the anatomical structure of interest includes at least one anatomical marker;
determining a rate of change in position of the at least one anatomical marker between adjacent image data sets;
calculating an acquisition frame rate based on the rate of change in position of the at least one anatomical marker; and
acquiring first and second image data sets at the acquisition frame rate.

2. The method of claim 1, further comprising calculating a second acquisition frame rate based on the rate of change in position of the at least one anatomical marker between second adjacent image data sets.

3. The method of claim 2, wherein the acquisition frame rate is different from the second frame rate.

4. The method of claim 1, wherein the acquisition frame rate include at least one data set, during the data set no ultrasound data is acquired.

5. The method of claim 1, further comprising identifying the at least one anatomical marker within the adjacent image data sets based on an image analysis algorithm, wherein the image analysis algorithm is defined using a machine learning algorithm.

6. The method of claim 1, wherein the ultrasound data is received along a wireless bi-directional communication link with an ultrasound probe.

7. The method of claim 6, further comprising adjusting a data throughput along the wireless bi-directional communication link based on the rate of change in position of the at least on anatomical marker.

8. The method of claim 1, wherein the rate of change represents a velocity of the at least one anatomical marker.

9. The method of claim 1, further comprising calculating a capacity of a power supply, and adjusting the acquisition frame rate based on the capacity.

10. The method of claim 1, wherein the anatomical structure of interest is a heart, a bladder, a kidney or a liver.

11. A mobile ultrasound imaging system comprising:
a portable host system having a controller circuit, a power supply, and a display, wherein the controller circuit is configured execute programmed instructions stored in a memory, the controller circuit when executing the programmed instructions perform the following operations:
receive ultrasound data representing image data sets of an anatomical structure of interest, wherein the anatomical structure of interest includes at least one anatomical marker;
determine a rate of change in position of the at least one anatomical marker between adjacent image data sets;
calculate an acquisition frame rate based on the rate of change in position of the at least one anatomical marker; and
acquire first and second image data sets at the acquisition frame rate.

12. The mobile ultrasound imaging system of claim 11, wherein the controller circuit is further configured to perform the following operation calculate a second frame rate based on the rate of change in position of the at least one anatomical marker between second adjacent image data sets.

13. The mobile ultrasound imaging system of claim 12, wherein the controller circuit is further configured to perform the following operation display third and fourth image data sets at the second frame rate.

14. The mobile ultrasound imaging system of claim 11, wherein the acquisition frame rate include at least one data set, during the data set no ultrasound data is acquired.

15. The mobile ultrasound imaging system of claim 11, wherein the controller circuit is further configured to perform the following operations identify the at least one anatomical marker within the adjacent image data sets based on an image analysis algorithm stored in the memory, wherein the image analysis algorithm is defined using a machine learning algorithm.

16. The mobile ultrasound imaging system of claim 11, further comprising a communication circuit, the communication circuit is configured to maintain a wireless bi-directional communication link with an ultrasound probe, wherein the ultrasound data is received along the wireless bi-directional communication link.

17. The mobile ultrasound imaging system of claim 16, wherein the controller circuit is further configured to perform the following operation adjust a data throughput along the wireless bi-directional communication link based on the acquisition frame rate.

18. The mobile ultrasound imaging system of claim 11 wherein the rate of change represents a velocity of the at least one anatomical marker.

19. The mobile ultrasound imaging system of claim 11, wherein the controller circuit is further configured to perform the following operation calculate a capacity of a power supply, and adjusting the acquisition frame rate based on the capacity.

20. A tangible and non-transitory computer readable medium comprising one or more programmed instructions configured to direct one or more processors to:
receive ultrasound data representing image data sets of an anatomical structure of interest, wherein the anatomical structure of interest includes at least one anatomical marker;
determine a rate of change in position of the at least one anatomical marker between adjacent image data sets;
calculate an acquisition frame rate based on the rate of change in position of the at least one anatomical marker; and
acquire first and second image data sets at the acquisition frame rate.
